(19) United States
(12) Patent Application Publication
(10) Pub. No.: US 2011/0269718 A1
(43) Pub. Date: Nov. 3, 2011

(54) PHARMACEUTICAL COMPOSITION CONTAINING MALATHION AND VOLATILE CYCLIC SILICONE

(75) Inventor: Michel Jeanjean, Castanet Tolosan (FR)

(73) Assignee: PIERRE FABRE DERMO-COSMETIQUE Corporation, Boulogne (FR)

(21) Appl. No.: 13/138,130

(22) PCT Filed: Jan. 11, 2010
(86) PCT No.: PCT/FR2010/050029

§ 371 Date: Jul. 11, 2011

(51) Int. Cl. A61K 31/661; A61P 33/14; C07F 9/201

(52) U.S. Cl. 514/122; 558/71

(57) ABSTRACT

The invention described comprises using volatile silicone oils in stabilising malathion. The invention also comprises pediculicidal and/or nit-killing compositions comprising malathion and volatile silicone oils and also excipients for topical use.

The present invention relates to a composition for use in the treatment of pediculosis, characterised in that it associates malathion with a volatile silicone oil, the latter serving as a solvent vehicle for the pediculicidal molecule.

Pediculosis is common and occurs throughout the world, affecting people of all ages and all socio-economic groups. However, contamination is most frequent between the ages of 3 and 10. Moreover, this contamination is very often asymptomatic.

Pediculus humanus var capitis De Geer, or the louse, is a cosmopolitan parasite exclusive to humans which lives in the hair on the head, away from which it survives for only 24 to 48 hours. The optimum survival temperature for the louse is between 28 and 30° Celsius. Contamination may occur directly (head-hair contact) or indirectly (sharing of combs, brushes, hats, scarves etc.). Even brief contact is sufficient for lice to pass from one person to another. A single heavily infested person may act as a reservoir for the parasites. A transmission risk factor is a large number of people living together in a restricted space.

Pediculosis with P. capitis is considered by some authors to be the most frequent contagious disease in schools after respiratory infections. According to the report by the Conseil Supérieur d'Hygiène de France (French Higher Council for Public Health) dated 17 Jan. 2003, the size of the problem caused by this disease is related simultaneously to the ease of contact transmission, to the repeated failures in treatment, to re-contamination after treatment and to the frequency of secondary infections (impetigo). In addition, the financial outlay of families in combating the parasite is sometimes considerable because the costs of pediculicides are not reimbursed by health systems, which may ultimately result in persistence of the epidemic.

It is moreover observed that pediculosis is undergoing a resurgence, one of the reasons for which could be a problem with resistance to organochlorines such as DDT and to pyrethrum derivatives.

The sign of an infection is the presence of nits in the hair and the presence of constant itching due to the bites, which correspond to blood meals. There is also a possibility of secondary infection of the scratched lesions.

There are two types of protection against the lice: mechanical-type protection and protection of the type involving application of a product directly to the hair. Mechanical-type protection consists of using a mechanical or electrical comb. These are not sufficiently effective because lice and nits that are not removed are still capable of reproducing. In addition, the lice and nits are difficult to remove using combs.

Some silicone derivatives also act by physical action; more specifically, hexamethylsiloxane, also known as dimethicone, when it dries, envelops the lice in a plastic film which accordingly blocks any possibility of respiration. Decamethylcyclo-pentasiloxane, also referred to as cyclomethicone, is sometimes used in conjunction therewith in order to improve spreading of the hexamethylsiloxane.

Protection consisting of the application of a product to the hair is based on use of an insecticide in shampoos, sprays or powders.

That pediculosis treatment involves using a variety of insecticidal molecules such as lindane (hexachlorocyclohexane), which is an organochlorine compound. Its use is indicated in pediculosis but it has been found to have no action on the nits. The side-effects of this compound are serious, as this compound can give rise to neurological toxicity which manifests itself in convulsions, as well as haematological toxicity in children less than 2 years old if systemic passage occurs.

Synthetic pyrethrin and pyrethroid compounds are also widely used in the context of treating pediculosis. This molecule also causes side-effects such as a smarting or burning sensation if it is applied to the eyes or mucous membranes. It is also contra-indicated if this molecule is used in pressurised canisters for patients suffering from asthma or having a history of dyspnoeic bronchitis.

Malathion is an organophosphorus compound and is well known as an insecticide. Malathion is a treatment of choice in pediculosis and, in addition, has excellent nit-killing action. This compound is in fact widely recognised as being the most active and constitutes the reference compound currently on the pediculicide market. Malathion may be used on its own or in combination with pyrethrins.

However, the vectorisation of malathion requires special measures because of its instability in conventional media, especially the polar solvents such as ethanol and isopropanol that are customarily used. In those customarily used solvents isomerisation of the malathion takes place, causing a partial reduction in its activity. The first step in the malathion degradation results in iso-malathion (see FIG. 1) being formed under the effect of temperature. The second step, in which iso-malathion is converted into other compounds, depends on several other physico-chemical factors.

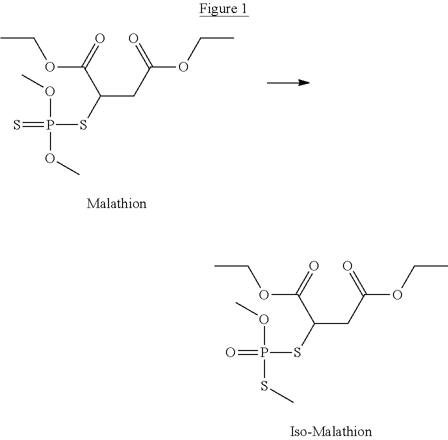

The present invention accordingly comprises stabilising the malathion by using volatile silicone oils. In the present invention, silicone oils are defined as polydimethylcyclosiloxanes and short-chain linear polysiloxanes or an association of polydimethylcyclosiloxanes and short-chain linear polysiloxanes. Preferably, the polydimethylcyclosiloxane is decamethylcyclopentasiloxane or cyclomethicone. Preferably, the short-chain linear polysiloxane is hexamethylsiloxane or dimethicone.

The present invention accordingly comprises using polydimethylcyclosiloxanes and more especially decamethylcyclopentasiloxane in stabilising malathion.

The present invention also comprises using short-chain linear polysiloxanes and more especially hexamethylsiloxane in stabilising malathion.

The present invention also comprises using volatile silicone oil in an amount of more than 50% in stabilising malathion.

The value of associating malathion with an apolar vector and more especially a volatile silicone oil is found on two levels:
the volatile silicone oil stabilises the naturally unstable malathion.
the volatile silicone oil, for example decamethylcyclopentasiloxane, itself has a pediculicidal activity of 100% and a valuable nit-killing activity of the order of 70%.

Volatile silicone oil accordingly constitutes the ideal solvent for vectorising malathion in a pediculicidal and/or nit-killing composition whilst ensuring its perfect stability. The volatile silicone oil moreover evaporates after a period of five hours and has a low viscosity making it easy to remove by rinsing. Preferably, the volatile silicone oil chosen for stabilising the malathion is selected from polydimethylcyclosiloxanes and short-chain polysiloxanes.

The invention relates also to a pediculicidal and/or nit-killing pharmaceutical and/or dermatological composition comprising malathion and volatile silicone oil in association with excipients for topical use.

The invention relates also to a pediculicidal and/or nit-killing pharmaceutical and/or dermatological composition comprising malathion stabilised by a volatile silicone oil with excipients for topical use.

The invention relates more especially to a pediculicidal and/or nit-killing composition containing, as two sole active ingredients, malathion and volatile silicone oil. The invention also comprises a composition containing:
from 0.1% to 10% malathion and from 40% to 99.9% volatile silicone oil.

Even more preferably, the invention comprises a composition containing:
from 0.1% to 0.5% malathion and from 99.5 to 99.9% volatile silicone oil.

Preferably, the volatile silicone oil used in the pediculicidal and/or nit-killing compositions is selected from polydimethylcyclosiloxanes and short-chain polysiloxanes.

The compositions according to the invention comprise malathion and a volatile silicone oil selected from polydimethylcyclosiloxanes and more especially decamethylcyclopentasiloxane.

The compositions according to the invention comprise malathion and a volatile silicone oil selected from short-chain polysiloxanes and more especially hexamethylsiloxane.

The formulation of the composition according to the invention is intentionally very simple, as inclusion of additives such as perfumes could cause destabilisation of the malathion.

The invention comprises compositions packaged in the form of sprays, mousses, gels or lotions.

The Examples that follow illustrate the invention without limiting its scope.

### EXAMPLE 1

### Stabilisation of Malathion by a Polydimethylcyclosiloxane

The inventors measured malathion degradation by HPLC. The HPLC method was reverse-phase HPLC with UV detection (210 nm). The column used is an xTERRA C18 with a water/acetonitrile gradient and a flow rate of 0.6 ml/min. The malathion compositions tested comprise either malathion on its own, or a composition of 50% malathion and 50% isopropanol, or a composition of 50% malathion and 50% polydimethylcyclosiloxane, or a composition of 33% malathion with 33% isopropanol and 33% polydimethylcyclosiloxane. These compositions also contain excipients such as povidone as viscosity agent.

The stability of these compositions was assessed for 114 hours at 60° Celsius (Table 1).

The results given in Table 1 show that:
a) malathion is much more stable in solution with polydimethylcyclosiloxane than with isopropanol.
b) the stability of malathion in association with polydimethylcyclosiloxane is greater than that of malathion on its own.
c) the stability of malathion in solution with isopropanol and polydimethylcyclosiloxane is significantly less than the stability of malathion in solution with polydimethylcyclosiloxane.

It is accordingly possible to conclude that isopropanol reduces the stability of malathion in solution whilst polydimethylcyclosiloxane improves it substantially.

TABLE 1
Stability of malathion over time.

| | | |
|---|---|---|
| | Duration in | |
| | hours and | Malathion |
| | Temperature in | content (%)/ |
| Composition | degrees Celsius | loss (%) |

| Malathion alone | T0 | 100.90 | |
|---|---|---|---|
| | T 72 hrs 60° C. | 100.3 | (−0.1) |
| | T 114 hrs 60° C. | 91 | (−9.8) |
| Malathion/isopropanol | T0 | 0.514 | |
| | T 72 hrs 60° C. | 0.485/(−5.6) | |
| | T114 hrs 60° C. | 0.461 | (−10.3) |
| Malathion/ | T0 | 0.508 | |
| polydimethylcylosiloxane | T 72 hrs 60° C. | 0.505 | (−0.6) |
| | T 114 hrs 60° C. | 0.507 | |
| Malathion/isopropanol/ | T0 | 0.509 | |
| polydimethylcyclosiloxane/ | T 72 hrs 60° C. | 0.495 | (−2.75) |
| Povidone | T 114 hrs 60° C. | 0.481 | (−5.5) |

### EXAMPLE 2

### Pediculicidal and Nit-Killing Activity of a Solution of Malathion and Decamethylcyclopentasiloxane

1) Pediculicidal Activity Test

This test was carried out on batches of 60 lice:
30 lice aged from 3 to 5 days
30 lice aged from 18 to 20 days.

For performance of this test, the batch of lice is placed in the bottom of a Petri dish having a diameter of 5 centimetres covered with filter paper, and 300 μl of test solution are then poured directly onto the lice. The lice are then left on the solution-impregnated filter paper and are observed after a period of one hour and after a period of 24 hours. The test solution is as follows:
0.5% malathion and 95.5% decamethylcyclopentasiloxane.

Results:

TABLE 2
Pediculicidal activity of the solution

| | Results | | | | | |
|---|---|---|---|---|---|---|
| Time | Dead lice | | Knocked-down lice | | Live lice | |
| of action | number | % | number | % | number | % |
| T0 | 38 | 63 | 22 | 37 | 0 | 0 |
| T 1 hr | 54 | 90 | 6 | 10 | 0 | 0 |
| T 24 hrs | 60 | 100 | 0 | 0 | 0 | 0 |

After a period of one hour, it is found that 90% of the lice are dead whilst the remaining 10% have been weakened (“knocked down”). After a period of 24 hours, 100% of the lice are dead. The solution combining malathion and decamethylcyclo-pentasiloxane accordingly has very efficacious pediculicidal activity.
2) Nit-Killing Activity Test

The nit-killing activity of this solution was also verified. For this purpose, the solution of 0.5% malathion and 99.5% decamethylcyclopentasiloxane was applied to the nits on day D0. The nits used are young, 1- to 2-day-old, nits and 8- to 9-day-old nits. Hatching of these nits was monitored for 14 days.

Results:

TABLE 3
Nit-killing activity of the solution

Hatchings after a
period of 14 days

| Age | Number of nits | number | % |
|---|---|---|---|
| young (1 to 2 days) | 80 | 0 | 0 |
| old (8 to 9 days) | 88 | 0 | 0 |

Whatever the age of the nits tested, the use of the solution comprising 0.5% malathion and 99.5% decamethylcyclopentasiloxane prevents hatching of all nits, in 100% of cases.

The solution comprising 0.5% malathion and 99.5% decamethylcyclo-pentasiloxane therefore has pediculicidal and nit-killing activity.

### EXAMPLE 3

### Formulations

Example of Lotion A
Malathion: 0.5%
Decamethylcyclopentasiloxane: 99.5%

Example of Lotion B
Malathion: 0.5%
Decamethylcyclopentasiloxane: 50%
Low-molecular-weight hydrocarbon compounds in liquid form (also referred to by the generic term “mineral oils”): 49.5%

Example of Atomiser Spray
Malathion: 0.5%
Hexamethyldisiloxane: 39.5%
Decamethylcyclopentasiloxane: 60%

Example of Aerosol Spray
Malathion: 0.5%
Decamethylcyclopentasiloxane: 40%
Low-molecular-weight hydrocarbon compounds in liquid form (also referred to by the generic term “mineral oils”): 59.5%
Mixture of hydrocarbon propellants q.s.

1-13. (canceled)

14. A method for stabilizing malathion, comprising adding a volatile silicone oil to the malathion.

15. The method of claim 14, wherein the volatile silicone oil is selected from polydimethylcyclosiloxanes, short-chain linear polysiloxanes, and mixtures thereof.

16. The method of claim 15, wherein the polydimethylcyclosiloxane is decamethylcyclopentasiloxane.

17. The method of claim 15, wherein the short-chain linear polysiloxane is hexamethylsiloxane.

18. The method of claim 14, wherein the volatile silicone oil is added to the composition comprising malathion in an amount greater than 50%.

19. A pediculicidal and/or nit-killing composition comprising malathion, volatile silicone oils, and excipients.

20. A pediculicidal and/or nit-killing composition consisting essentially of malathion and volatile silicone oil.

21. The pediculicidal and/or nit-killing composition of claim 19, wherein the silicone oil is selected from polydimethylcyclosiloxanes, short-chain linear polysiloxanes, and mixtures thereof.

22. The pediculicidal and/or nit-killing composition of claim 21, wherein the polydimethylcyclosiloxane is decamethylcyclopentasiloxane.

23. The pediculicidal and/or nit-killing composition of claim 21, wherein the short-chain linear polysiloxane is hexamethylsiloxane.

24. The pediculicidal and/or nit-killing composition of claim 19, wherein the malathion is present between 0.1% and 10%, and wherein the volatile silicone oil is present between 40% and 99.9%.

25. The pediculicidal and/or nit-killing composition of claim 19, wherein the malathion is present between 0.1% and 0.5%, and wherein the volatile silicone oil is present between 99.5% and 99.9%.

26. The pediculicidal and/or nit-killing composition of claim 19, which is in the form of sprays, mousses or lotions.
